(19) United States
(12) Patent Application Publication
(10) Pub. No.: US 2023/0337953 A1
(43) Pub. Date: Oct. 26, 2023

(54) INCONTINENCE DETECTION

(71) Applicant: I-Innovations Ltd., Belfast (GB)

(72) Inventors: John Kelly, Tyrone (IE); Iain Warner, New Alresford (GB)

(73) Assignee: I-Innovations Ltd., Belfast (GB)

(21) Appl. No.: 17/729,744

(22) Filed: Apr. 26, 2022

(51) Int. Cl. A61B 5/20; A61B 5/01

(52) U.S. Cl. CPC A61B 5/202; A61B 5/01; A61B 5/4255

(57) ABSTRACT

The present invention relates to a computer implemented method for controlling a gas detector, a gas detector, a control system for detecting an incontinence event, and a computer implemented method for detecting an incontinence event. The method for controlling a gas detector comprises receiving a plurality of datasets from the gas detector. Each dataset is associated with a time interval and comprises at least one environmental measurement; and at least one detected level of ammonia. The method comprises receiving one or more user measurements, where a user measurement comprises an incontinence state determined at a particular time. The method comprises determining criteria to identify an incontinence event, using the received plurality of datasets and the received user measurement; and transmitting the criteria to the gas detector. The criteria define a characteristic of a subset of data of the plurality of datasets indicating an incontinence event.

### FIELD OF INVENTION

This invention relates to a computer implemented method, at a computing device, for controlling a gas detector and a gas detector.

### BACKGROUND

There is a global increase in the strain associated with world health care economies; a key contributor to this increased strain is the aging population′ and increase in care management that is associated with taking care of the elderly. A common care requirement associated with the aging population is incontinence care and continence promotion.

Incontinence, the inability to control one's urine or bowel movements, is most common among older peoples, those with dementia, and other faculty diminishing conditions; it is also a major driving force in long-term care admissions.

At least half of the elderly population within Britain's and North America's care homes suffer from incontinence.

Although care homes are often portrayed as a safe haven for our most vulnerable population, many authorities have dissented from the view that they are a continence-friendly environment. When incontinence is not attended to, a person can experience degeneration of his/her skin health, making him/her prone to pressure ulcers and bedsores in regions of the body that remain in contact with the faecal matter or urine. Since many elderly individuals are incapacitated and cannot summon assistance, this means caretakers or the like must be relied on to firstly detect and then respond promptly to the problem. As the proportion of older people in populations increases worldwide and the number of care homes is increasing, there is the requirement for improved independent living assistive technology.

Currently, in care homes, incontinence management may rely on a manual process of checking for incontinence events, whereby it is the responsibility of a member of the nursing care team to detect the characteristic odour of diapers/pants that need to be changed. This can be difficult to monitor on a 24-hour basis particularly late at night or in remote parts of the care home. This can also result in a sense of reduced dignity for many suffers, especially those who still uphold many of their mental faculties.

Throughout this specification, in relation to the present invention, the term “incontinence” is to be understood as including both faecal and urinary incontinence. Furthermore, the term “soiling” is to be understood to refer to soiling by urine and soiling by faeces.

### BRIEF SUMMARY OF THE DISCLOSURE

In accordance with the present inventions there is provided a method for controlling a gas detector, comprising: receiving datasets from the gas detector, each dataset associated with a time interval and comprising: at least one measurement; and at least one detected level of a gas; receiving a user measurement comprising an incontinence state determined at a particular time; determining criteria to identify an incontinence event, using the received datasets and the received user measurement; and transmitting the criteria to the gas detector; wherein: the criteria define a characteristic of a subset of data of the plurality of datasets indicating an incontinence event.

In this way, the method of the disclosure provides comprehensive and accurate understanding of background non-incontinence associated environmental measurements, such as levels of ammonia and other gases, as well as incontinence-associated environmental measurements related to an incontinent person, such as ammonia etc values within the environment.

In accordance with the present inventions there is provided a computer implemented method, at a computing device, for controlling a gas detector, comprising: receiving a plurality of datasets from the gas detector, each dataset associated with a time interval and comprising: at least one environmental measurement; and at least one detected level of ammonia; receiving one or more user measurements, the or each measurement comprising an incontinence state determined at a particular time; determining criteria to identify an incontinence event, using the received plurality of datasets and the or each received user measurement; and transmitting the criteria to the gas detector; wherein: the criteria define a characteristic of a subset of data of the plurality of datasets indicating an incontinence event.

Optionally, the plurality of datasets is received from a plurality of gas detectors; receiving one or more user measurements comprises receiving multiple user measurements with each of the multiple user measurements comprising an incontinence state determined at a particular time for a particular gas detector of the plurality of gas detectors; and the transmitted criteria is transmitted to at least a subset of the plurality of gas detectors.

In an example, the criteria are applied to a time series of detected levels of ammonia, and the criteria comprise a lower threshold and an upper threshold of a rate of increase of the level of ammonia between adjacent readings in the time series.

Optionally, the criteria further comprise a pattern in the time series and a retry parameter, wherein determining if the pattern is present in the time series comprises skipping measurements in the time series if the number of consecutive measurements skipped does not exceed the retry parameter. In this way, incontinence detection performance is improved, by mitigating unknown brief events that result in ambient air being directed away from the gas detector, for example if a window was opened.

In an example, the one or more user measurements comprises one or more user measurements acquired through a user interface of an application on a portable electronic communication device.

Optionally, the plurality of datasets comprises: one or more datasets that have been transmitted in response to an incontinence event being detected; and datasets that have been transmitted in response to reporting conditions being satisfied, the reporting conditions being independent of whether an incontinence event has been detected.

In an example, determining criteria to identify an incontinence event, using the plurality of datasets and the or each user measurement, comprises: processing the plurality of datasets in a learning module using the or each user measurement as a source of truth to determine the criteria.

Optionally, determining criteria to identify an incontinence event, using the plurality of datasets and the or each user measurement, comprises: filtering the plurality of datasets to identify a subset of the plurality of datasets corresponding to a particular setting; and retrieving a subset of the one or more user measurements corresponding to the subset of the plurality of datasets; determining the criteria to identify an incontinence event in the particular setting using the subset of the plurality of datasets and the subset of the one or more user measurements; transmitting the criteria to at least a subset of the plurality of gas detectors comprises transmitting the criteria to the subset of the plurality of gas detectors.

In an example, the particular setting is infant care. The particular setting may include a residential/domestic care environment, a health care facility, nursing home and other care environments.

In accordance with a further aspect of the present inventions, there is provided a gas detector configured to: obtain measurements from sensors, each measurement associated with a time; generate datasets from the measurements, each dataset associated with a time interval and comprising: an environmental measurement; and a detected level of a gas; transmit the datasets to a computing device; receive, from the computing device, criteria that define a characteristic of a subset of data of the datasets indicating an incontinence event; and determine whether an alert should be raised by assessing if the received criteria are satisfied.

In accordance with another aspect of the present inventions, there is provided a gas detector configured to: obtain measurements from a plurality of sensors, each measurement associated with a time; generate a plurality of datasets from the measurements, each dataset associated with a time interval and comprising: at least one environmental measurement; and at least one detected level of ammonia; transmit the plurality of datasets to a computing device; receive, from the computing device, criteria that define a characteristic of a subset of data of the plurality of datasets indicating an incontinence event; and determine whether an alert should be raised by assessing if the received criteria are satisfied.

Optionally, the gas detector has memory for storing measured data, the memory being of a size which is insufficient to retain the data of the plurality of datasets.

In an example, the criteria are applied to a time series of detected levels of ammonia, and the criteria comprise a lower threshold and an upper threshold of a rate of increase of the level of ammonia between adjacent readings in the time series.

Optionally, the criteria further comprise a pattern in the time series and a retry parameter, wherein determining if the pattern is present in the time series comprises skipping measurements in the time series if the number of consecutive measurements skipped does not exceed the retry parameter.

The gas detector may further comprise a user interface and is further configured to transmit, to the computing device, one or more user measurements acquired through the user interface, the or each measurement indicating an incontinence state determined at a particular time.

Optionally, the gas detector is configured to transmit the datasets in the plurality of datasets: in response to an incontinence event being detected; and in response to reporting conditions being satisfied, the reporting conditions being independent of whether an incontinence event has been detected.

### BRIEF DESCRIPTION OF THE DRAWINGS

Embodiments of the invention are further described hereinafter with reference to the accompanying drawings, in which:

FIG. 1 is a flow chart of a method according to an embodiment of the invention;

FIG. 2 is a block diagram of a system according to an example of the disclosure;

FIG. 3 is a block diagram of a further system according to an example of the disclosure;

FIG. 4 is a block diagram of another system according to an example of the disclosure;

FIG. 5 is a block diagram of a gas detector according to an embodiment of the invention;

FIG. 6 is a flow diagram of a method for detecting an incontinence event according to an embodiment of the invention; and

FIG. 7 is a block diagram of a control system for detecting an incontinence event according to an embodiment of the invention.

### DETAILED DESCRIPTION

The present invention will now be described more fully hereinafter with reference to the accompanying drawings, in which embodiments of the invention are shown. This invention may, however, be embodied in many different forms and should not be construed as limited to the embodiments set for the herein. Rather, these embodiments are provided so that this disclosure will be thorough and complete, and will fully convey the scope of the invention to those skilled in the art.

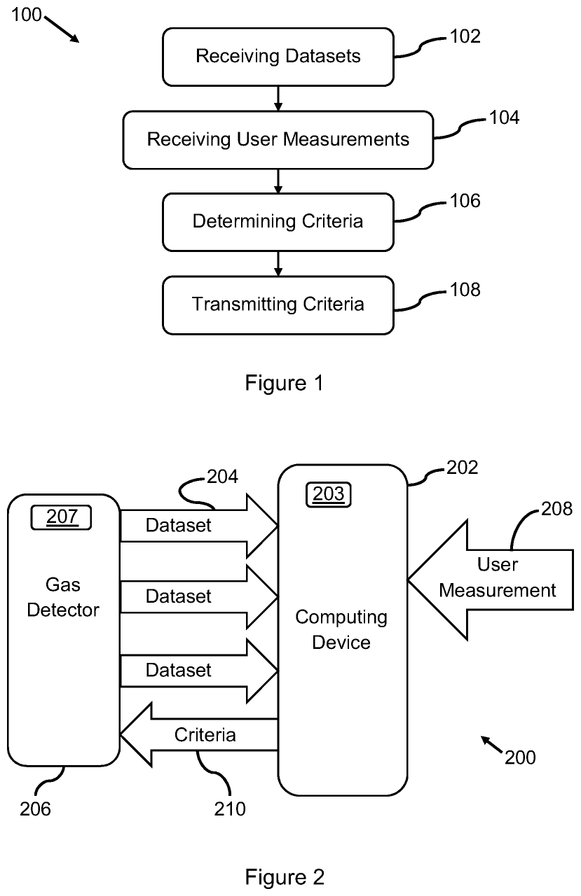

Referring to FIGS. 1 and 2, there is shown a method, indicated generally by the reference numeral 100, for controlling a gas detector, and a system, indicated generally by the reference numeral 200 in which the method may be implemented. The method 100 is a computer-implemented method, implemented by a computing device 202. The computing device 202 further comprises a processor 203 as described herein. The method comprises receiving 102 a plurality of datasets 204 from a gas detector 206. The gas detector 206 further comprises one or more sensors 207 as described herein. Each dataset 204 is associated with a time interval and comprises at least one environmental measurement; and at least one detected level of ammonia. Typically, the computing device 202 receives the datasets 204 in a sequential manner.

The method comprises receiving 104 one or more user measurements 208, the or each user measurement 208 comprising an incontinence state determined at a particular time. The computing device 202 is adapted to receive the one or more user measurements 208. An incontinence state may comprise information relating to the incontinent person, for example, that there has been no incontinence event, that there has been a fecal incontinence event, that there has been a urinary incontinence event or that that there has been a combined fecal and urinary incontinence event. The user measurement may also indicate the degree of the incontinence.

The method comprises determining 106 criteria 210 to identify an incontinence event, using the received plurality of datasets 204 and the received user measurement 208. The criteria 210 define a characteristic of a subset of data of the plurality of datasets 204 indicating an incontinence event.

The method comprises transmitting 108 the criteria 210 to the gas detector 206.

The user measurements 208 may be received from the gas detector 206, but are not limited thereto. Any source of user measurements 208 relating to an incontinence state at a particular time may be used, for example a dedicated user device or a mobile application on a portable electronic communications device.

The environmental measurement may comprise at least one measurement of a temperature; a pressure; and a humidity or the like of the ambient environment of at least one of the gas detectors 206. Ambient air temperature and humidity are useful in determining criteria having increased accuracy for the identification of an incontinence event.

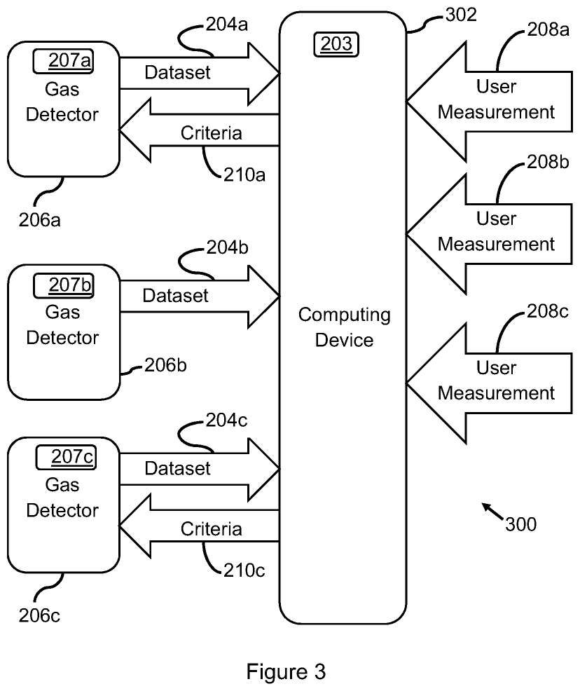

Referring now to FIG. 3, there is shown a block diagram of an alternative system, indicated generally by the reference numeral 300, in which the method 100 described above may operate. A care home setting may comprise a plurality of incontinent persons, typically each of whom would have a corresponding gas detector, for example located on or beside their bed. The system 300 comprises a plurality of gas detectors 206a, 206b, 206c, each of which may send datasets 204a, 204b, 204c respectively to the computing device 202. Note each to the gas detectors 206a, 206b, 206c further comprises one or more sensors 207a, 207b, 207c. In this way, the plurality of datasets 204a, 204b, 204c is received from a plurality of gas detectors 206a, 206b, 206c. The computing device 302 of the system 300 is adapted to receive multiple user measurements. Each of the multiple user measurements comprises an incontinence state determined at a particular time for a particular gas detector 206 of the plurality of gas detectors 206a, 206b, 206c. The criteria 210a, 210c are transmitted to at least a subset of the plurality of gas detectors, in the system 300 of FIG. 3, the subset comprises two gas detectors 206a, 206c. In this way, the computing device 202 receives datasets relating to the plurality of incontinent persons and provides criteria for determining for incontinence events to the appropriate gas detectors. The use of multiple gas detectors allows for data aggregation from a number of environments, and so provides for improved determination of the criteria to identify an incontinence event. In this way, criteria for a particular setting may be adjusted based on past performance in similar settings. Additionally, this allows for identifying causes of conflict e.g. it might be that there is a link between high-humidity or temperature and false-positives. In such an example, in cold conditions, it may be useful to adjust the configurable criteria.

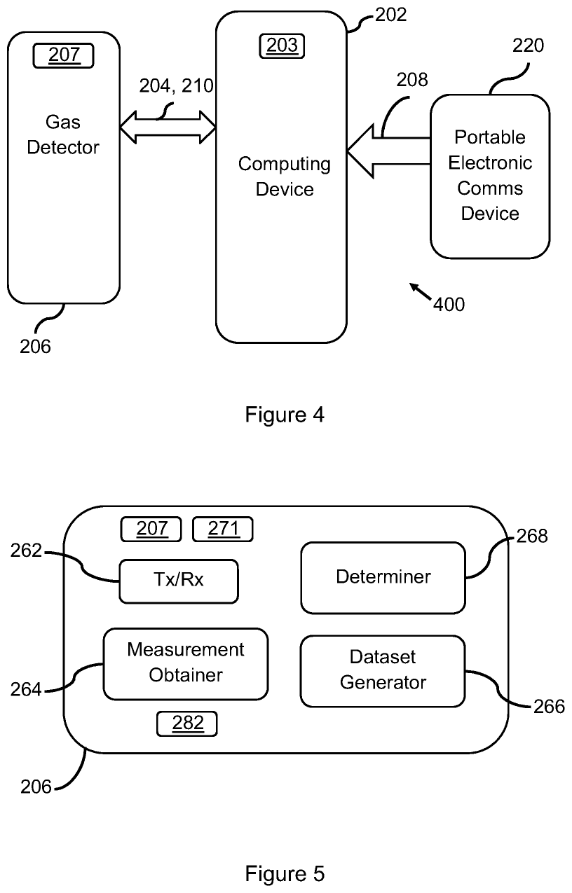

Referring now to FIG. 4, there is shown a further system, indicated generally by the reference numeral 400, in which the method 100 may operate. The system 400 is similar to those described herein in relation to FIG. 2 or 3 and comprises a gas detector 206 and computing device 202. The system further comprises a portable electronic communication device 220. The portable electronic communications device 220 is configured to transmit a user measurement 208 to the computing device 202. The user measurement may be entered by a user of the system, for example a caretaker or the like. The system 400 of FIG. 4 may be combined with the system 300 of FIG. 3, such that the system may comprise a plurality of gas detectors 206, and may further comprise a plurality of portable electronic communications devices 220 for providing user measurements to the computing device 202. While it is understood that a portable electronic communication device is convenient for users, it is not necessary for the device to be a personal or portable device. A desktop or laptop computer or like device that can accept user inputs may be used to provide the user measurements.

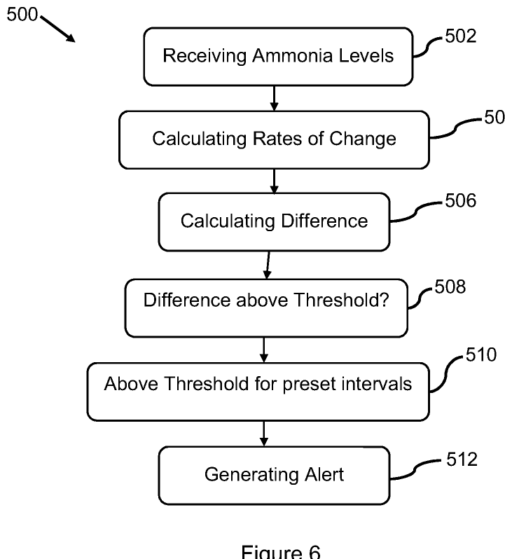

Referring now to FIG. 5, there is shown a block diagram of a gas detector 206 according to an example of the disclosure. The gas detector 206 comprises a communications module 262, which may be adapted to transmit and receive data from external devices, such as the computing device 202. The gas detector 206 further comprises a measurement obtainer 264, configured to obtain measurements from a plurality of sensors 207, where each measurement is associated with a time. The gas detector 206 may include one or more suitable sensors 207 or the sensors 207 may be separate from the gas detector 206. The gas detector 206 further comprises a dataset generator 266. The dataset generator 266 is configured to generate a plurality of datasets from the measurements. Each dataset is associated with a time interval and comprises at least one environmental measurement; and at least one detected level of ammonia. The datasets may be transmitted to the computing device 202 by the communications module 262. The gas detector 206 is configured to receive, from the computing device 202, criteria that define a characteristic of a subset of data of the plurality of datasets indicating an incontinence event. The gas detector 206 is configured to receive the criteria via the communications module 262. The gas detector 206 further comprises a determiner 268 configured to determine whether an alert should be raised by assessing if the received criteria are satisfied. The measurement obtainer 264, dataset generator 266 and determiner 268 may be implemented by one or more controllers, processors or the like, as will be understood by the skilled person.

The sensors 207 associated with the gas detector 206 may comprise an ammonia sensor. It will be understood that absolute values of ammonia levels may vary across use settings and may be in a constant state of change both concerning the background (non-incontinent associated) ammonia levels and the marker (incontinent associated) event levels. The ammonia sensor may be chosen to have a sensing range that will provide useful sensor readings at a useable distance from the source of ammonia. For example, a sensor can detect changes in ammonia levels when located on a wall behind a bed-ridden incontinent person's bed. The inventors have discovered that an ammonia sensor with a minimum of 150 ppm NH₃ sensitivity range is adequate to detect incontinence event ammonia levels from a distance of at least 60 cms. In examples according to the present disclosure, an ammonia sensor with a 500 ppm minimum NH₃ sensitivity may be used to provide for a broad scope of options with positioning the ammonia sensors at increased distances from the incontinent person.

The ammonia sensor may be referred to as an ambient gas sensing or “sniffer” device. The ammonia sensor may be located close to the anatomical mid-section of the incontinence sufferer. The sensor used may be structured so that the gas supply is limited by diffusion and thus the output from the sensor is linearly proportional to the gas concentration. Such as linear output allows greater control over the measurement of low concentrations and much simpler calibration and cross referencing with pre-set standards. In an example, the gas detector comprises a plurality of ammonia sensors.

The gas detector 206 may comprise an enclosure casing for housing the components described in relation to FIG. 5 (the casing not being depicted for ease of illustrating the components therein), and any sensors 207 if the sensors form part of the gas detector 206. In an example where the gas detector 206 comprises the sensors 207, the enclosure casing may be adapted to encourage airflow intake while reducing internal pressure inside the enclosure housing so as to ensure that the sensors function optimally.

Suitable devices include MICS-5914, MICS-6814, MICS-5524 from SGS Sensortech. The sensors associated with the gas detector 206 may comprise a humidity sensor and temperature sensor. In an example, the sensors comprise a combined humidity and sensor temperature. The SHTC₃ sensor from Sensirion is a suitable device. The sensors may further include sensors for measuring other gases such as methane, hydrogen, carbon monoxide, carbon dioxide, nitrogen dioxide, and Volatile Organic Compounds (VOCs). Considering environmental measurements and a variety of gases allows for distinguishing between incontinence events more accurately, and reducing false positive alarms due to environmental conflicts. Considering broad spectrum VOCs allows for further insights in relation to environmental conflicts which may have similar ammonia profiles to the incontinence events but detectable differences in broader gaseous emission profiles. The Bosch BME680 may be a suitable sensor to provide VOC data.

The gas detector 206 may further comprise memory 282 for storing measured data. However, in an example, the memory 282 is of a size which is insufficient to retain the data of the plurality of datasets. In this way, the cost and device size resulting from significant quantities of data storage can be avoided. Some measured data may be stored on the gas detector 206, but may be overwritten as new data is received. The measured data may be stored at the computing device.

The gas detector 206 may further comprises a user interface (not shown). The user interface may be configured to allow a user to enter user measurements. Such user measurements indicate an incontinence state determined at a particular time.

An incontinence state may be that a urinary incontinence event has occurred, that a fecal incontinence event has occurred, that a combined incontinence event has occurred or that no incontinence event has occurred. The communication module may transmit user measurements 208 so acquired to the computing device.

The communications module 262 may be adapted to implement a communication protocol suitable to communicate with the computing device. The communication between the gas detector 206 and the communications device may be direct, or may be indirect, for example, via the Internet. An installation of the system in a particular setting described herein may further comprise a central controller (not shown). The gas detectors 206 may communicate with the computing device 202 via the central controller. The communications module 262 may allow communication via Wi-Fi, or other suitable method. The gas detector 206 may be configured as an Internet of Things device, and may use appropriate communications protocols.

The computing device 202 may comprise a server or other suitable computing device. The computing device 202 may be local to the care setting in which the gas detectors are located or may be remote therefrom. The computing device 202 may be implemented as a cloud-based server. In an example, the computing device is implemented using the Microsoft Azure platform.

In use, a gas detector 206 obtains readings in relation to its surroundings. These readings may be obtained from suitable sensors, which may be part of the gas detector 206 or may be external to the gas detector and in communication therewith. The sensors may measure ambient conditions, such as environmental conditions and gas levels including ammonia levels. The environmental conditions may include temperature and humidity. Readings from the sensors, which may be referred to samples, are taken regularly, to provide substantially continual measurement of the variables of interest. In an example, the sensors are configured to sample once every 1 to 10 seconds, such as every second, every two seconds or every five seconds. The sampling rate is configurable, and is not limited to the examples provided herein. Adjustments to the sampling rate may lead to changes in detection threshold levels, as a longer time between samples may result in larger increases in ammonia levels between samples.

The various readings are combined into a dataset. In one example, one dataset is a set of one ammonia level, one temperature and one humidity. However, it will be understood that other dataset configurations are within the scope of the present disclosure. For example, an alternative dataset configuration may comprise only an ammonia level and a temperature, or a further example dataset may comprise an ammonia level, a temperature, a humidity, a methane level and a carbon monoxide level.

The gas detector 206 transmits the datasets 204 from the various sensors to the computing device 202. This reduces the data storage requirement for the gas detector 206, and also facilitates processing and analysis of the datasets 204 at the computing device 202. The datasets 204 may be transmitted in response to certain reporting conditions being satisfied. Example reporting conditions may be scheduled times, timed intervals, pseudo-randomly, when local memory (e.g., the memory 282) of the gas detector 206 is full, and the like. The reporting conditions may be independent of whether an incontinence event has been detected. The reporting conditions, and the manner and settings in which the gas detector 206 provides datasets 204 to the computing device 202 may be configurable by a system user, for example, configurable for each care setting installation or depending on use case.

In an example, the gas detector 206 is configured to transmit a dataset 204 corresponding to each group of sensor readings. In this way, a dataset may be transmitted, for example, once every 1 to 10 seconds, such as every second, every two seconds or every five seconds. This may be referred to as a “heartbeat” signal.

While the heartbeat signal is useful in providing environment data to the computing device 202, is it also useful in indicating that the gas detector 206 is functioning. An absent or disrupted heartbeat message indicates an issue with a gas detector 206, and the computing device 202 can indicate that maintenance of the device may be required.

Datasets may also be transmitted to the computing device 202 in response to an incontinence event being detected.

The computing device 202 also receives user measurements relating to an incontinence state or event in the vicinity of the gas detector 206. Typically, each gas detector 206 in a care setting will be associated with a single incontinent person of the care setting. For example, for bed-ridden incontinent persons, a gas detector 206 may be located on or adjacent to their bed. The user measurement may be entered via a user interface on the gas detector or computing device, or via a further device such as a portable electronic communication device including a tablet or smartphone device.

Incontinence alarms from the gas detector 206 can be used to indirectly trigger the generation of a user measurement. When an alarm is generated, a staff member of the care setting will attend the incontinent person for whom the alert has been generated and then create a user measurement indicating the incontinence state of the incontinent person. However, it will be understood that user measurements may be entered at any time and it is not required that they be entered in response to an alarm from the gas detector. For example, the gas detector may fail to detect an incontinence event, which is later identified by a caretaker or the like. The caretaker or the like may generate a user measurement to indicate that there had been an incontinence event.

The user measurement may indicate the type of incontinence event. The user measurement may also indicate that there has not in fact been an incontinence event, even though an incontinence alert may have been raised by a gas detector 206. In this way, the user measurement may be used as a source of truth in relation to incontinence events. False positives may be a result of ground operations at the setting in which the gas detector 206 is operating, for example cleaning products and methodology of cleaning.

When a user measurement indicates that an incontinence event has taken place, this information is combined with the dataset or datasets for the appropriate time.

In this way, a training set of data is provided to the computing device 202 for use in determining criteria for use by the gas detector to detect incontinence events with improved accuracy. This training set of data includes datasets from when there was no incontinence event to affect the readings.

Using this collected data, the computing device 202 is able to determine criteria suitable for identifying incontinence events based on readings from the sensors of environmental measurements and gas levels including ammonia levels. The computing device 202 then transmits the criteria to the gas detector 206 so that the gas detector 206 may apply the criteria to the datasets obtained at the gas detector 206.

The computing device 202 may use a learning module to determine the criteria to identify an incontinence event. The learning module may implement machine learning techniques to analyse the datasets, including any associated user measurements, and determine the criteria to identify an incontinence event. In this way, the criteria to detect an incontinence event can be improved and adapted to conditions. For example, where there is residual odor in a room, the criteria can be specified to identify such a condition, and the relevant threshold level, e.g. ammonia levels, can be adjusted to avoid false positive alarms. In another example, the criteria can be adjusted in light of the cleaning and ventilation conditions in effect in a room, such that the relevant thresholds can be adjusted. The learning module may be configured to process the received user measurements, cross-check these with the datasets and their associated times to search for correlation and degree of correlation with a view to improve performance via adjusting configurable detection logic parameters. The learning module, which may also be referred to as a machine learning classification model, may utilise machine learning techniques such as Random Forest or Neural Network methods or other suitable techniques. The machine learning techniques may be supported by an Active Learning process to determine criteria for identifying incontinence events based on the datasets received from the gas detector and the user measurements.

Through the confirmation and refutation of incontinence alerts raised by the gas detector(s) 206, and the confirmation of an incontinence event type by the user, the system can build a data point catalogue of ambient ammonia data trends and associated system sensing values that are, and are not, indicative of incontinent events. Based on this data point catalogue, the computing device 202, 302 can autonomously refine and tune the detection logic parameters based on the confirmation user inputs fed into the system by the caretaker or the like.

The gas detector 206 maintains a time series of detected levels of ammonia, and a time series of the sensed environmental measurements. The time series of detected levels comprises the received datasets. In the case of other measurements that may be captured, for example, levels of other gases, a time series of these measurements may also be maintained. The various time series may fully correspond, for example, for a time point in the one time series having a data measurement, each of the other time series will also have a data measurement for that point. However, such correspondence is not required, and it may be the case that a time series only has a sparser set of data measurements such that certain timepoints will not have a measurement associated therewith.

The computing device 202, 302 receives datasets from the gas detector 206 or gas detectors. The datasets may be considered as comprising two types of dataset. Firstly, there is the heartbeat datasets as discussed previously. These are sent at regular intervals to provide substantially continuous readings to the computing device 202. These datasets comprise at least one environmental measurement and at least one detected level of ammonia. The computing device may also receive event datasets. Event datasets relate to an incontinence event and/or incontinence alert, and may be transmitted in response to an incontinence event being detected and/or an incontinence alert being raised. Event datasets comprise at least one environmental measurement; and at least one detected level of ammonia, and may also comprise processed versions of this data, for example, baseline ammonia levels, average ammonia levels, rate of change of the ammonia levels, and rate of change of the average ammonia levels. Other variables such as the environmental measurements may also be processed in this way. Event datasets comprise data from before, during, and after the event in question.

An incontinence event may be identified by an increase in the ammonia level in the environment. The rate of increase of the ammonia level is a useful indicator of an incontinence event. The criteria may comprise specifying a lower threshold and/or an upper threshold of a rate of increase of the level of ammonia between adjacent readings in the time series.

The criteria 210 determined by the computing device may specify a pattern to appear the time series data. The criteria 210 may also specify a retry parameter, wherein determining if the pattern is present in the time series comprises skipping measurements in the time series if the number of consecutive measurements skipped does not exceed the retry parameter.

Determining the criteria to identify an incontinence event may be carried out to create criteria for specific settings, specific user types, and so on. For determining criteria for a specific setting, the computing device 202 may filter the datasets to identify a subset thereof corresponding to the setting of interest. The computing device 202 may also retrieve corresponding user measurements. The computing device 202 may retrieve all of the corresponding user measurements or a selection thereof. The criteria to identify an incontinence event in the particular setting can then be determined using the subset of datasets and the selection of corresponding user measurements. The determined criteria may then be transmitted to the relevant gas detectors. In some cases, the determined criteria may be sent to all gas detectors to be available for each gas detector, or the determined criteria may be sent to the gas detectors associated with the specific setting.

The gas detector 206 may begin operation using initial criteria, which are then updated with the determined criteria from the computing device 202. Alternatively, the gas detector 206 may begin operation with no initial criteria and will only generate incontinence alarms after the determined criteria have been received. In this situation, the users would provide user measurements every time an incontinence event occurs, and also preferably when no incontinence event has occurred, to allow the relevant data to be flagged.

The gas detector 206 may begin operation with predetermined criteria to identify an incontinence event stored thereon. These predetermined criteria may be referred to as initial criteria. The predetermined criteria may be stored in the memory 282 on the gas detector 206. In this way, the gas detector may trigger an incontinence alarm before receiving criteria from the computing device 202. The sample data from the ammonia sensor may be processed and analysed by the gas detector 206 and, if the criteria are met, an incontinence alarm is raised. The processing may comprise calculating an average ammonia level over time and the rate of change of the ammonia levels. The rate of change may then be compared to a threshold to determine if an alarm should be raised. The gas detector 206 provides the data that led to the alarm being raised to the computing device 202. This data may be provided in the form of an event dataset. This alarm data is in addition to the heartbeat data, which is transmitted substantially continually.

In an example, the initial criteria are configured to generate an incontinence alarm when a continual increase in ammonia levels over time is detected in the ambient environment. The gas detector 206 stores ammonia level readings at the gas detector 206. The ammonia levels may be stored in a buffer or the like, such that only a certain number of recent values are stored. The readings, which may be referred to as samples, from the ammonia sensors, may be taken at the same time and/or rate as the heartbeat signal is to be transmitted. In this way, each ammonia level in the buffer may be taken from the same reading as the data included in the datasets 204 forming the heartbeat signal. In an example, sixty ammonia levels are stored in the buffer. When the buffer is full, the oldest values may be over-written with the newer data. A rolling average of the ammonia levels stored in the buffer may be calculated. The rolling average values may also be stored, for example in a similar buffer. The rate of change of the ammonia levels may be calculated based on this average. The rate of change may be calculated using a first order derivative. The values for the rate of change may also be stored, for example in a further buffer. If the rate of change is above a predefined threshold for a predefined number of readings, then an incontinence alarm can be raised by the gas detector 206. When the alarm is raised, the ammonia levels buffer, average ammonia levels buffer and first order derivate values buffer are sent to the computing device 202, as an event dataset.

In this way, the computing device 202 is receiving the heartbeat signal comprising ammonia levels and one or more environmental measurement, and also a data package of ammonia levels, averages and first order derivatives for each alarm raised by the gas detector 206. Furthermore, the computing device 202 receives a user measurement 208 indicating the incontinence state of the incontinent person at that time. The user measurement 208 may be considered as a source of truth as to the in relation to the incontinence state of the incontinent person. As such, the computing device 202 has accurate information of the incontinence state of the incontinent person, the data that led to the incontinence alarm being raised; and data including environmental measurements from before, during and after the alarm event. The computing device 202 analyses all of this data to determine updated criteria for triggering an incontinence alarm, and transmits those criteria to the gas detectors 206. The updated criteria may include changed values for the predefined threshold and the predefined number of readings.

While the description describes alerts being triggered by the gas detector 206 based on locally stored data configurable criteria which may be adjusted by instructions from the computing device, it will be understood, that alert triggering may be carried out by the computing device if data can be transmitted from the gas sensors and environmental measures and to the gas detectors in a suitably fast manner, with due consideration of the power, data storage, and date processing requirements.

Referring now to FIG. 6, there is shown a flow diagram of a method, indicated generally by the reference numeral 500, for detecting an incontinence event. The method may be implemented at a computing device such as the computing device 202 or gas detector 206 described herein. The method 500 comprises receiving 502 a series of detected ammonia levels from a sensor, each detected ammonia level being associated with a time interval. At block 504, the method comprises calculating rates of change of the ammonia level based on the series of detected ammonia levels. At block 506, the method comprises calculating the difference between rates of change that are a preset period of time apart. At block 508, the method comprises determining if the difference is above a first threshold value. At block 504, the method comprises generating an incontinence alert indicating a likely incontinence event, if, for repeated calculations of the rates of change, the difference is above the first threshold value for a first preset number of time intervals.

Calculating a rate of change may comprise calculating a first order derivative. Calculating the first order derivative may comprise determining an average ammonia level based on the plurality of detected ammonia levels. This may be referred to as a baseline ammonia level. In an initial operation phase of the method, a number of detected ammonia levels may be collected before an average value is calculated, for example 20 to 100 values. In a particular example, over 45 values are collected. As each new detected ammonia level is received, the average level is recalculated. This may be referred to as a windowed average. Once the desired number of detected ammonia levels have been collected, newer detected levels will overwrite the oldest collected samples.

A sample detected ammonia level is also obtained, as may be used as part of calculating the first order derivative. The sample detected ammonia level may be the most recently received detected ammonia level.

For the first order derivative, the difference between the average ammonia level and the sample detected ammonia level may be calculated. The difference may be adjusted by a gain constant to provide a gain value. This may comprise multiplying the difference by a gain constant, but other adjustments may be used. The gain constant may be configurable. In an example, the gain constant is 2.

The first order derivative is calculated from the difference between gain values a second preset number of time intervals apart.

If the difference is not above the first threshold, the method 500 may comprise skipping time intervals in the first preset number of time intervals if the number of consecutive measurements skipped does not exceed a retry parameter.

Each of the time intervals, the preset period of time, the first threshold value, the first preset number of time intervals, the gain constant, the retry parameter and the second preset number of time intervals are configurable, and appropriate adjustments to these configurable settings may be included in the criteria received from the computing device 202. However, it will be understood that the criteria determined by the computing device are not limited to these settings. In this way, if the gas detector 206 is failing to generate incontinence alerts in response to incontinence events identified by the user, the detection logic can be refined through the computing device providing updated criteria. These updated criteria may comprise, for example, increasing the gain constant which may help amplify output; increasing the retry parameter which may account for ambient airflow volatility; and/or reducing the first threshold value and the second preset number of time intervals which may allow more subtle changes in ammonia levels to be detected. The opposite can be applied if the gas detector initially generates too many false positive incontinence alerts.

In an example of the invention of the disclosure, the method described in relation to FIG. 6 is implemented by a gas detector 206 described herein. The criteria provided by the computing device 202 may comprise values or settings for one or more of the time intervals, the preset period of time, the first threshold value, the first preset number of time intervals, the gain value, and the second preset number of time intervals.

In an example, where detected ammonia levels are received every 2 seconds, with a gain constant of 2, the first threshold value may be between 10 and 20 and typically between 13 and 17. The first preset number of the time intervals may be between 15 and 25 intervals. In this way, every two seconds each sample collected must be e.g. 17 ppm higher than the preceding value for a total of e.g. 25 collected samples.

The retry parameter is applicable in instances where a detected value is less than that what preceded, and gives flexibility to avoid a situation of missing an incontinence event because of a small disturbance in a slope of continuous rising ammonia values. Such a disturbance could be caused for example by a door or window being opened, or other such events where airflow to the sensor may be affected.

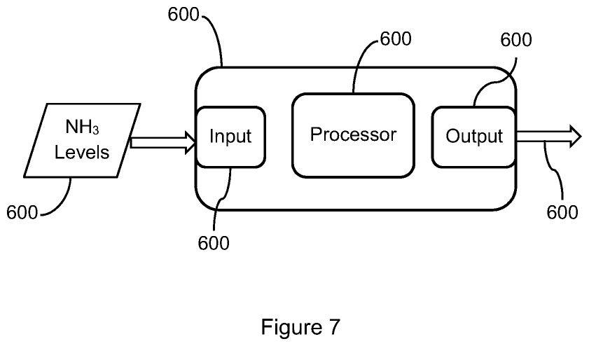

Referring now to FIG. 7, there is shown a block diagram of a control system 600 suitable for implementing the method described in relation to FIG. 6. The control system 600 comprises an input 602 for receiving a series of detected ammonia levels 604 from a sensor (not shown), each detected ammonia level being associated with a time interval. The control system 600 further comprises an output 606 for outputting an incontinence alert signal. The control system 600 further comprises a processor 610. The processor 610 is arranged to calculate rates of change of the ammonia level based on the series of detected ammonia levels. The processor 610 is further arranged to calculate the difference between rates of change that are a preset period of time apart. The processor 610 is further arranged to determine if the difference is above a first threshold value. The processor 610 is further arranged to generate an incontinence alert signal indicating a likely incontinence event; if, for repeated calculations of the rates of change, the difference is above the first threshold value for a first preset number of time intervals, generating an incontinence alert signal indicating a likely incontinence event.

In this way, the method 500 described in relation to FIG. 6 and the control system described in relation to FIG. 7, may be understood to implement incontinence detection based on detecting a substantially constant rate of change of ammonia levels or increasing rate of change of ammonia levels.

The system and methods described herein may also be configured to generate alerts based on the temperature readings, humidity readings and readings from other sensors associated with the gas detector 206.

Furthermore, through enabling user-confirmation of incontinence event type, the systems, devices and methods of the disclosure can quickly and accurately make predictions relating to an individual's future bowel and/or bladder movements. A deep learning sequence labelling algorithm can evaluate historic incontinence events to determine frequency irregularities with bowel and/or bladder movements and cross-references any determined frequency irregularities with a pre-set symptom database or comorbidities to uncover potential comorbidity risks.

The present disclosure relates to an apparatus and system for non-invasive incontinence detection, analysis and transmission.

The methods, systems and devices described herein are suitable for use in settings where the service-users or residents may require incontinence care, such as care homes. Other settings where incontinence care is relevant are infant care settings, including domestic setting and commercial infant care.

The user inputs can be received into the system through either a) an input on the gas detector or b) via a mobile application. The user measurements may comprise a missed incontinence event or may confirm whether the gas detector alerted correctly to incontinence, and if so, the type of incontinence e.g. FI, UI, FI+UI, type of FI. This confirmation of alert and alert type by the user acts to label the gas detector's alert data-set buffer. By having this multifaceted environment associated data set, the gas detectors can be configured for improved performance in a variety of specific client and environmental conditions.

The user measurements confirm whether the gas detector missed an incontinence event, or accurately alerted to an incontinence event. In this way, feedback is provided on the current configurable settings of the gas detector, indicating that current settings are functioning adequately or that adjustment may be useful.

The heartbeat signal in addition to letting a remote admin know the device is working, also provides an ongoing time series log of ambient conditions which can a) be used to correspond with user measurements, for example retrospectively analyse data relating to an incontinence event that a caretaker or the like confirms but was missed by the gas detector. The heartbeat datasets, when combined with the alert datasets, provide a full signature diffusion profile over time, whereas the alert datasets provide time-limited diffusion data based around when an alert is triggered. Understanding the diffusion signature in full i.e. base through to apex through to base may be useful in providing for increased accuracy in detecting incontinence events.

As well as the gas levels and environmental measurements included in the datasets, they may also include time stamp, device id and other such information.

The incontinence detection logic of the disclosure does not require detection of absolute values of ammonia or breaches in finite ammonia thresholds, but rather seeks a specific pattern of the ammonia levels, for example a learned event signature diffusion trend. Embodiments of the disclosure may generate an alert when continual increase in the ammonia over time is detected from ambient air odours from human waste.

Certain aspects of the disclosure may be implemented using machine-readable instructions which may, for example, be executed by a general purpose computer, a special purpose computer, an embedded processor or processors of other programmable data processing devices to realize the functions described in the description and diagrams. In particular, a processor or processing apparatus may execute the machine-readable instructions. Thus, functional modules of the apparatus and devices may be implemented by a processor executing machine readable instructions stored in a memory, or a processor operating in accordance with instructions embedded in logic circuitry. The term ‘processor’ is to be interpreted broadly to include a CPU, processing unit, ASIC, logic unit, or programmable gate array etc. The methods and functional modules may all be performed by a single processor or divided amongst several processors.

The portable electronic communication device described in the present disclosure may include a personal communication device such as a mobile phone, a tablet device or the like; and/or other electronic devices such as a Portable Multimedia Player (PMP), a mobile medical device, a camera, or a wearable device such as a smart watch or smart glasses, a smart home appliance such as a home automation device, a security control panel, a TV box such as Apple TV™, or Google TV™), a gaming console (e.g., Xbox™, PlayStation™) or the like. The portable electronic communication device may include one of or a combination of the above-listed devices. The portable electronic communication device devices are not limited to the above-listed devices, and may include new electronic devices depending on the development of technology.

It will be appreciated that embodiments of the present invention can be realised in the form of hardware, software or a combination of hardware and software. Any such software may be stored in the form of volatile or non-volatile storage such as, for example, a storage device like a ROM, whether erasable or rewritable or not, or in the form of memory such as, for example, RAM, memory chips, device or integrated circuits or on an optically or magnetically readable medium such as, for example, a CD, DVD, magnetic disk or magnetic tape. It will be appreciated that the storage devices and storage media are embodiments of machine-readable storage that are suitable for storing a program or programs that, when executed, implement embodiments of the present invention. Accordingly, embodiments provide a program comprising code for implementing a system or method as claimed in any preceding claim and a machine-readable storage storing such a program. Still further, embodiments of the present invention may be conveyed electronically via any medium such as a communication signal carried over a wired or wireless connection and embodiments suitably encompass the same.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

Further examples of the present disclosure are set out in the following numbered clauses.
1. A computer implemented method, at a computing device, for controlling a gas detector, comprising:
   receiving a plurality of datasets from the gas detector, each dataset associated with a time interval and comprising:
      at least one environmental measurement; and
      at least one detected level of ammonia;
   receiving one or more user measurements, the or each measurement comprising an incontinence state determined at a particular time;
   determining criteria to identify an incontinence event, using the received plurality of datasets and the or each received user measurement; and
   transmitting the criteria to the gas detector;
wherein:
   the criteria define a characteristic of a subset of data of the plurality of datasets indicating an incontinence event.
2. The computer implemented method of clause 1, wherein:
   the plurality of datasets is received from a plurality of gas detectors;
   receiving one or more user measurements comprises receiving multiple user measurements with each of the multiple user measurements comprising an incontinence state determined at a particular time for a particular gas detector of the plurality of gas detectors; and
   the transmitted criteria is transmitted to at least a subset of the plurality of gas detectors.
3. The computer implemented method of clause 1 or 2, wherein the criteria is applied to a time series of detected levels of ammonia, and the criteria comprise a lower threshold and an upper threshold of a rate of increase of the level of ammonia between adjacent readings in the time series.
4. The computer implemented method of clause 3, wherein the criteria further comprise a pattern in the time series and a retry parameter, wherein determining if the pattern is present in the time series comprises skipping measurements in the time series if the number of consecutive measurements skipped does not exceed the retry parameter.
5. The computer implemented method of any preceding clause, wherein the one or more user measurements comprises one or more user measurements acquired through a user interface of an application on a portable electronic communication device.
6. The computer implemented method of any preceding clause, wherein the plurality of datasets comprises:
   one or more datasets that have been transmitted in response to an incontinence event being detected; and
   datasets that have been transmitted in response to reporting conditions being satisfied, the reporting conditions being independent of whether an incontinence event has been detected.
7. The computer implemented method of any preceding clause, wherein determining criteria to identify an incontinence event, using the plurality of datasets and the or each user measurement, comprises:
   processing the plurality of datasets in a learning module using the or each user measurement as a source of truth to determine the criteria.
8. The computer implemented method of any preceding clause, wherein:
   determining criteria to identify an incontinence event, using the plurality of datasets and the or each user measurement, comprises:
      filtering the plurality of datasets to identify a subset of the plurality of datasets corresponding to a particular setting; and
      retrieving a subset of the one or more user measurements corresponding to the subset of the plurality of datasets;
      determining the criteria to identify an incontinence event in the particular setting using the subset of the plurality of datasets and the subset of the one or more user measurements;
   transmitting the criteria to at least a subset of the plurality of gas detectors comprises transmitting the criteria to the subset of the plurality of gas detectors.
9. The computer implemented method of clause 8, wherein the particular setting is infant care.
10. A gas detector configured to:
   obtain measurements from a plurality of sensors, each measurement associated with a time;
   generate a plurality of datasets from the measurements, each dataset associated with a time interval and comprising: at least one environmental measurement; and
      at least one detected level of ammonia;
      transmit the plurality of datasets to a computing device;
   receive, from the computing device, criteria that define a characteristic of a subset of data of the plurality of datasets indicating an incontinence event; and
   determine whether an alert should be raised by assessing if the received criteria are satisfied.
11. The gas detector of clause 10, wherein the gas detector has memory for storing measured data, the memory being of a size which is insufficient to retain the data of the plurality of datasets.
12. The gas detector of clause 10 or 11, wherein the criteria is applied to a time series of detected levels of ammonia, and the criteria comprise a lower threshold and an upper threshold of a rate of increase of the level of ammonia between adjacent readings in the time series.
13. The gas detector of clause 12, wherein the criteria further comprise a pattern in the time series and a retry parameter, wherein determining if the pattern is present in the time series comprises skipping measurements in the time series if the number of consecutive measurements skipped does not exceed the retry parameter.
14. The gas detector of any one of clauses 10 to 13, wherein the gas detector further comprises a user interface and is further configured to transmit, to the computing device, one or more user measurements acquired through the user interface, the or each measurement indicating an incontinence state determined at a particular time.
15. The gas detector of any one of clauses 10 to 14, wherein the gas detector is configured to transmit the datasets in the plurality of datasets:
   in response to an incontinence event being detected; and
   in response to reporting conditions being satisfied, the reporting conditions being independent of whether an incontinence event has been detected.
16. A computer implemented method, at a computing device, for detecting an incontinence event, the method comprising
   receiving a series of detected ammonia levels from a sensor, each detected ammonia level being associated with a time interval;
   calculating rates of change of the ammonia level based on the series of detected ammonia levels;
   calculating the difference between rates of change that are a preset period of time apart;
   determining if the difference is above a first threshold value; and
   if, for repeated calculations of the rates of change, the difference is above the first threshold value for a first preset number of time intervals, generating an incontinence alert indicating a likely incontinence event.
17. The method of clause 16 wherein calculating a rate of change comprises calculating a first order derivative.
18. The method of clause 17 wherein calculating a first order derivative comprises determining an average ammonia level based on the plurality of detected ammonia levels;
   obtaining a sample detected ammonia level, and calculating the difference between the average ammonia level and the sample detected ammonia level;
   adjusting the difference by a gain constant to provide a gain value; and
   calculating a difference between gain values a second preset number of time intervals apart as the first order derivative.
   19. The method of clause 18 wherein the average ammonia level is determined using a windowed average of the plurality of detected ammonia levels.
20. The method of clause 19 wherein the window is greater than forty-five measurements.
21. The method of clause 16 comprising,
   if the difference is not above the first threshold, skipping time intervals in the first preset number of time intervals if the number of consecutive measurements skipped does not exceed a retry parameter.
22. The method in clause 21 wherein at least one of the time intervals, preset period of time, first threshold value, first preset number of time intervals, gain value, and second preset number of time intervals are configurable.
23. A control system for detecting an incontinence event, the control system comprising one or more controllers, the control system comprising:
   an input for receiving a series of detected ammonia levels from a sensor, each detected ammonia level being associated with a time interval;
   a processor arranged to
      calculate rates of change of the ammonia level based on the series of detected ammonia levels,
      calculate the difference between rates of change that are a preset period of time apart,
      determine if the difference is above a first threshold value; and
      if, for repeated calculations of the rates of change, the difference is above the first threshold value for a first preset number of time intervals, generate an incontinence alert signal indicating a likely incontinence event; and
   an output for outputting the incontinence alert signal.

1. A computer implemented method, at a computing device, for controlling a gas detector, comprising:
receiving a plurality of datasets from the gas detector, each dataset associated with a time interval and comprising:
at least one environmental measurement; and
at least one detected level of ammonia;
receiving one or more user measurements, the or each measurement comprising an incontinence state determined at a particular time;
determining criteria to identify an incontinence event, using the received plurality of datasets and the or each received user measurement; and
transmitting the criteria to the gas detector;
wherein:
the criteria define a characteristic of a subset of data of the plurality of datasets indicating an incontinence event.

2. The computer implemented method of claim 1, wherein:
the plurality of datasets is received from a plurality of gas detectors;
receiving one or more user measurements comprises receiving multiple user measurements with each of the multiple user measurements comprising an incontinence state determined at a particular time for a particular gas detector of the plurality of gas detectors; and
the transmitted criteria is transmitted to at least a subset of the plurality of gas detectors.

3. The computer implemented method of claim 1, wherein the criteria is applied to a time series of detected levels of ammonia, and the criteria comprise a lower threshold and an upper threshold of a rate of increase of the level of ammonia between adjacent readings in the time series.

4. The computer implemented method of claim 3, wherein the criteria further comprise a pattern in the time series and a retry parameter, wherein determining if the pattern is present in the time series comprises skipping measurements in the time series if the number of consecutive measurements skipped does not exceed the retry parameter.

5. The computer implemented method of claim 1, wherein the one or more user measurements comprises
one or more user measurements acquired through a user interface of an application on a portable electronic communication device.

6. The computer implemented method of claim 1, wherein the plurality of datasets comprises:
one or more datasets that have been transmitted in response to an incontinence event being detected; and
datasets that have been transmitted in response to reporting conditions being satisfied, the reporting conditions being independent of whether an incontinence event has been detected.

7. The computer implemented method of claim 1, wherein determining criteria to identify an incontinence event, using the plurality of datasets and the or each user measurement, comprises:
processing the plurality of datasets in a learning module using the or each user measurement as a source of truth to determine the criteria.

8. The computer implemented method of claim 1, wherein:
determining criteria to identify an incontinence event, using the plurality of datasets and the or each user measurement, comprises:
filtering the plurality of datasets to identify a subset of the plurality of datasets corresponding to a particular setting; and
retrieving a subset of the one or more user measurements corresponding to the subset of the plurality of datasets;
determining the criteria to identify an incontinence event in the particular setting using the subset of the plurality of datasets and the subset of the one or more user measurements;
transmitting the criteria to at least a subset of the plurality of gas detectors comprises transmitting the criteria to the subset of the plurality of gas detectors.

9. The computer implemented method of claim 8, wherein the particular setting is infant care.

10. A gas detector configured to:
obtain measurements from a plurality of sensors, each measurement associated with a time;
generate a plurality of datasets from the measurements, each dataset associated with a time interval and comprising:
at least one environmental measurement; and
at least one detected level of ammonia;
transmit the plurality of datasets to a computing device;
receive, from the computing device, criteria that define a characteristic of a subset of data of the plurality of datasets indicating an incontinence event; and
determine whether an alert should be raised by assessing if the received criteria are satisfied.

11. The gas detector of claim 10, wherein the gas detector has memory for storing measured data, the memory being of a size which is insufficient to retain the data of the plurality of datasets.

12. The gas detector of claim 10, wherein the criteria is applied to a time series of detected levels of ammonia, and the criteria comprise a lower threshold and an upper threshold of a rate of increase of the level of ammonia between adjacent readings in the time series.

13. The gas detector of claim 12, wherein the criteria further comprise a pattern in the time series and a retry parameter, wherein determining if the pattern is present in the time series comprises skipping measurements in the time series if the number of consecutive measurements skipped does not exceed the retry parameter.

14. The gas detector of claim 10, wherein the gas detector further comprises a user interface and is further configured to transmit, to the computing device, one or more user measurements acquired through the user interface, the or each measurement indicating an incontinence state determined at a particular time.

15. The gas detector of claim 10, wherein the gas detector is configured to transmit the datasets in the plurality of datasets:
in response to an incontinence event being detected; and
in response to reporting conditions being satisfied, the reporting conditions being independent of whether an incontinence event has been detected.

16. A computer implemented method, at a computing device, for detecting an incontinence event, the method comprising
receiving a series of detected ammonia levels from a sensor, each detected ammonia level being associated with a time interval;
calculating rates of change of the ammonia level based on the series of detected ammonia levels;
calculating the difference between rates of change that are a preset period of time apart;
determining if the difference is above a first threshold value; and
if, for repeated calculations of the rates of change, the difference is above the first threshold value for a first preset number of time intervals, generating an incontinence alert indicating a likely incontinence event.

24. The method of claim 16 wherein calculating a rate of change comprises calculating a first order derivative.

25. The method of claim 17 wherein calculating a first order derivative comprises
determining an average ammonia level based on the plurality of detected ammonia levels;
obtaining a sample detected ammonia level, and calculating the difference between the average ammonia level and the sample detected ammonia level;
adjusting the difference by a gain constant to provide a gain value; and
calculating a difference between gain values a second preset number of time intervals apart as the first order derivative.

26. The method of claim 18 wherein the average ammonia level is determined using a windowed average of the plurality of detected ammonia levels.

27. The method of claim 19 wherein the window is greater than forty-five measurements.

28. The method of claim 16 comprising,
if the difference is not above the first threshold, skipping time intervals in the first preset number of time intervals if the number of consecutive measurements skipped does not exceed a retry parameter.

29. The method of claim 21 wherein at least one of the time intervals, preset period of time, first threshold value, first preset number of time intervals, gain value, and second preset number of time intervals are configurable.

30. A control system for detecting an incontinence event, the control system comprising one or more controllers, the control system comprising:
an input for receiving a series of detected ammonia levels from a sensor, each detected ammonia level being associated with a time interval;
a processor arranged to
calculate rates of change of the ammonia level based on the series of detected ammonia levels,
calculate the difference between rates of change that are a preset period of time apart,
determine if the difference is above a first threshold value; and
if, for repeated calculations of the rates of change, the difference is above the first threshold value for a first preset number of time intervals, generate an incontinence alert signal indicating a likely incontinence event; and
an output for outputting the incontinence alert signal.
